(19) United States
(12) Patent Application Publication
(10) Pub. No.: US 2016/0100774 A1
(43) Pub. Date: Apr. 14, 2016

(54) BIOMETRIC DEVICE

(71) Applicant: Young Living Essential Oils, LC, Lehi, UT (US)

(72) Inventors: Matt Wilcox, Bryan, TX (US); Andrew Robbins, Bryan, TX (US); Taraz Nosrat, Houston, TX (US); Samantha Miller, Friendswood, TX (US); Christine Fung, New York, NY (US)

(21) Appl. No.: 14/879,064

(22) Filed: Oct. 8, 2015

Related U.S. Application Data
(60) Provisional application No. 62/061,595, filed on Oct. 8, 2014.

(51) Int. Cl. A61B 5/097; A61B 5/00; A61B 5/08

(52) U.S. Cl. CPC A61B 5/097; A61B 5/082; A61B 5/7271; A61B 2560/0443

(57) ABSTRACT

Disclosed herein is one embodiment of a biometric device that includes a housing, a sensor, and a controller. The housing has a main chamber and a breath chamber. The breath chamber has an inlet and an outlet. The sensor is coupled to the main chamber and is at least partially in fluid contact with the breath chamber. The sensor is configured to detect the concentration of at least one gas of a user's breath in the breath chamber. The controller is coupled to the housing and is disposed within the main chamber. The controller is configured to receive the concentration of the at least one gas in the user's breath in the breath chamber and determine a health score.

### FIELD

The subject matter of the present disclosure relates generally to a biometric device, and more particularly relates to a biometric device for determining a health score of a user based on the user's breath.

### BACKGROUND

Tracking human characteristics of a user's body is an important aspect of health improvement and health maintenance. Indeed, monitoring one's bodily characteristics is useful in tracking the beneficial results of a certain treatment or determining the effect of certain lifestyle behaviors.

### SUMMARY

From the foregoing discussion, it should be apparent that a need exists for an apparatus, system, and method for detecting and determining a health indicator that overcome the limitations of conventional tools and procedures. Beneficially, such an apparatus, system, and method would improve the ease, efficiency, and effectiveness of measuring and tracking a user's health score.

The subject matter of the present application has been developed in response to the present state of the art, and in particular, in response to the problems and needs in the art that have not yet been fully solved by currently available health testing tools. Accordingly, the present disclosure has been developed to provide an apparatus, system, and method that overcome many or all of the above-discussed shortcomings in the art.

Disclosed herein is one embodiment of a biometric device. The biometric device includes a housing, a sensor, and a controller. The housing has a main chamber and a breath chamber. The breath chamber has an inlet and an outlet. The sensor is coupled to the main chamber and is at least partially in fluid contact with the breath chamber. The sensor is configured to detect the concentration of at least one gas of a user's breath in the breath chamber. The controller is coupled to the housing and is disposed within the main chamber. The controller is configured to receive the concentration of the at least one gas in the user's breath in the breath chamber and determine a health score.

In one implementation, the at least one gas includes carbon monoxide. In such an implementation, the at least one gas further includes carbon dioxide. The device may further include an inlet valve and a moisture filter coupled to the inlet of the breath chamber of the housing. Both the inlet valve and the moisture filter may be configured to be in fluid communication with the breath of the user. In one implementation, the inlet valve is a one way valve that prevents the breath from exiting the breath chamber through the inlet. The device may further include an outlet valve that is a one way valve that prevents atmosphere from entering the breath chamber through the outlet.

In one implementation, the main chamber and the breath chamber of the housing are detachable from each other. In another implementation, the controller includes a breath chamber module configured to indicate a status of the breath chamber. In another implementation, the controller has a valve module configured to indicate a status of valves disposed in the inlet and outlet of the breath chamber. In yet another implementation, the controller includes a sensor module configured to determine the concentration of the at least one gas of the breath of the user in the breath chamber. In such an implementation, the controller may include a data module configured to determine the health score based on the concentration of the at least one gas of the breath of the user. In another implementation, the controller includes the breath chamber module, the valve module, the sensor module, and the data module.

Also disclosed herein is one embodiment of a biometric system. The biometric system includes a housing, a sensor, and a controller. The housing has a main chamber and a breath chamber. The breath chamber has an inlet and an outlet. The sensor is coupled to the main chamber and is at least partially in fluid contact with the breath chamber. The sensor is configured to detect the concentration of at least one gas of a user's breath in the breath chamber. The controller includes a breath chamber module, a valve module, a sensor module, and a data module.

In one implementation, the controller is integrated on a computer removed from the housing. In another implementation, the system may further include an inlet valve and a moisture filter coupled to the inlet of the breath chamber of the housing. Both the inlet valve and the moisture filter may be configured to be in fluid communication with the breath of the user. In one implementation, the inlet valve is a one way valve that prevents the breath from exiting the breath chamber through the inlet. The system may further include an outlet valve that is a one way valve that prevents atmosphere from entering the breath chamber through the outlet.

Also disclosed herein is one embodiment of a method for determining a health score of a user. The method includes positioning a mouthpiece of a biometric device into the mouth of the user and exhaling a breath through the mouthpiece an into a breath chamber of the biometric device. The method also includes detecting the presence and concentration of at least one gas in the breath of the user and determining a health score of the user based on the presence and concentration of the at least one gas in the breath of the user.

In one implementation, the method includes holding the breath within the breath chamber for a predetermined time before allowing the breath to exit the breath chamber.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the subject matter disclosed herein. Thus, discussion of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize that the subject matter of the present application may be practiced without one or more of the specific features or advantages of a particular embodiment. In other instances, additional features and advantages may be recognized in certain embodiments that may not be present in all embodiments of the disclosure. Further, in some instances, well-known structures, materials, or operations are not shown or described in detail to avoid obscuring aspects of the subject matter of the present disclosure. These features and advantages of the present disclosure will become more fully apparent from the following description and appended claims, or may be learned by the practice of the disclosure as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention, and are not therefore to be considered to be limiting of its scope, the invention will be described and explained with additional specificity and detail through the use of the accompanying drawings, in which:

FIG. 1 is a schematic cross section view of a biometric device, according to one embodiment;

FIG. 2 is a front view of the biometric device, according to one embodiment;

FIG. 3 is a schematic block diagram of one embodiment of a controller for determining a health score of a user; and

FIG. 4 is a schematic flow chart diagram of one embodiment of a method for determining a health score of a user.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the disclosure may be combined in any suitable manner in one or more embodiments. In the following description, numerous specific details are provided. One skilled in the relevant art will recognize, however, that the disclosure may be practiced without one or more of the specific details, or with other methods, components, materials, and so forth. In other instances, well-known structures, materials, or operations are not shown or described in detail to avoid obscuring aspects of the disclosure.

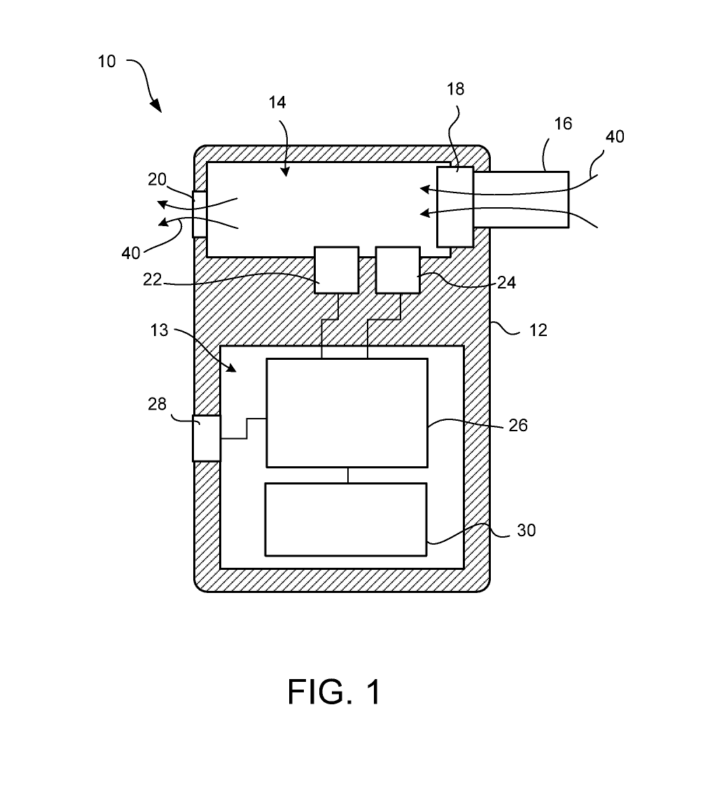

Reference will now be made to FIG. 1, which illustrates a cross section view of a biometric device 10, according to some embodiments. The biometric device 10 can be configured to measure carbon monoxide (CO), carbon dioxide (CO2), and/or other compounds from a breath sample 40. For example, the biometric device 10 receives a breath sample 40 as a user breathes into the device. The biometric device 10 measures one or more of the compounds within the breath sample 40 in order to determine a health status of the user or a relative health status of the user. For example, the biometric device 10 may be used to determine whether the user has been smoking or used to determine the levels of inflammation in the body of the user. Such determinations by the biometric device 10 are used to determine one or more health indicators (e.g., a health score) and/or one or more other biometrics. In the depicted embodiment, the biometric device 10 includes a device housing 12 that defines an internal chamber 14. The biometric device 10 also includes an inlet mouthpiece 16, a filter 18, and an outlet valve 20 that are coupled to the device housing 12 and that are in fluid transferring communication with the internal chamber 14 (described in greater detail below). The biometric device 10 also includes a first sensor 22, a second sensor 24, a processor 26, a power source 28, and a display 30. In some embodiments, one or more of these components are omitted from the biometric device. For example, the second sensor 24 may be omitted and the first sensor 22 may be used to measure the compounds within a breath sample 40 of the user. Also, the display 30 may be omitted in some embodiments (e.g., in one implementation, the sensors 22, 24 may directly communicate results to a removed computer or network for analysis).

The device housing 12 may be a device shell, case, complete enclosure, or other suitable structure. The device housing 12 retains and protects the internal components (e.g., circuitry, processor 26, power source 28, display element 30, etc.) of the biometric device 10 inside or partially inside the device housing 12. Additionally, fluid components, such as the inlet mouthpiece 16, the filter 18, and the outlet valve 20, may be coupled to the device housing 12. As described above, in some embodiments the device housing 12 forms and defines an internal chamber (i.e., “breath chamber 14”). In another embodiment, however, the breath chamber may not be housed within the device housing 12 but instead may be externally disposed, relative to the device housing 12. In one embodiment, the device housing 12 also defines a second chamber (i.e., a main chamber 13) that houses at least the processor 26 and display 30. In other embodiments, the device housing 12 forms multiple internal chambers to house the multiple, internal components.

For example, the device housing 12 can form a chamber, such as a main chamber 13 that contains the processor 26, power source 28, and/or the display 30. The main chamber 13 can be designed to allow the display 30 and any associated interface buttons (not shown) to extrude through holes formed through the wall(s) of the housing 12 so that the user can easily see the display 30 and interact with the buttons. The main chamber 13 may also contain at least a portion of the first sensor 22 and second sensor 24 and is in fluid communication with the mouthpiece 16 via the filter 18. One or more holes may be formed through the walls of the main chamber 13 permit a portion of the first sensor 22 and second sensor 24 to extend into the breath chamber 14. All other components within the main chamber 13 can be positioned so that they are fully internal to the main chamber 13. In one embodiment, the back of the main chamber 13 is removable so that components can be easily positioned inside.

In one embodiment, the main chamber 13 and the breath chamber 14 are detachable from each other. In other words, the breath chamber 14 may be detached from the main chamber 13 in order to store the breath sample for a period of time. In another implementation, the breath chamber 14 may be disposable and may thus be configured for one time use. In yet another embodiment, the breath chamber 14 may be detached from the main chamber 13 to be cleaned or sanitized, etc., before being reattached and reused with the main chamber 13.

The housing 12 may be formed of various materials, including metal, polymer, plastic, and other suitable materials. For example, in one embodiment, the housing 12 is formed, at least in part, of ABS plastic, which provides reasonable robustness while still remaining lightweight. The housing 12 may have various dimensions. In some embodiments, the housing 12 is shaped and sized to be held in the average adult human hand, rendering the biometric device 10 as a handheld device. In other embodiments, the housing 12 may have other shapes and sized. For example, the housing 12 may have an overall length of is approximately 30 cm, a width of approximately 20 cm, and a height is approximately 30 cm. In some embodiments, the housing 12 can include an access port, removable panel, door, or other device for permitting access to the components housed within the housing 12.

As mentioned, in some embodiments, the biometric device 10 includes an inlet mouthpiece 16 that is configured and positioned to introduce a breath sample 40 into the breath chamber 14. The inlet mouthpiece 16 can be integrated with the housing 12 or may be disposable/replaceable. For example, the inlet mouthpiece 16 can be a breathalyzer mouthpiece from AK GlobalTech Corporation.

In one embodiment, the mouthpiece 16 contains a one-way check valve 17 which allows for one-directionality of flow. The check valve 17 may be designed to remain closed when the pressure on the device end of the mouthpiece 16 is equal to or higher than the pressure on the patient end of the mouthpiece 16. When the pressure on the patient end of the mouthpiece 16 is higher than that of the device end of the mouthpiece, the check valve 17 opens to allow for airflow. The check valve 17 operates close to ideally and requires only a very small opening pressure to open. This amount of pressure is easily provided by even a minimal force breath.

Upon use, the inlet mouthpiece 16 is configured to be positioned with the patient end outside the device 10 and the device end being in fluid communication with the breath chamber 14 via the filter. This allows breath 40 to enter the breath chamber 14 when the patient breathes into the device but prevents the escape of breath 40 out of the breath chamber 14. The inlet mouthpiece 16 may be easily removable and may be coupled to the breath chamber hole snugly but not tightly so that it can be easily replaced. Though the inlet mouthpiece 16 is durable, it may be replaced whenever a new patient uses the device for hygiene/sanitary purposes.

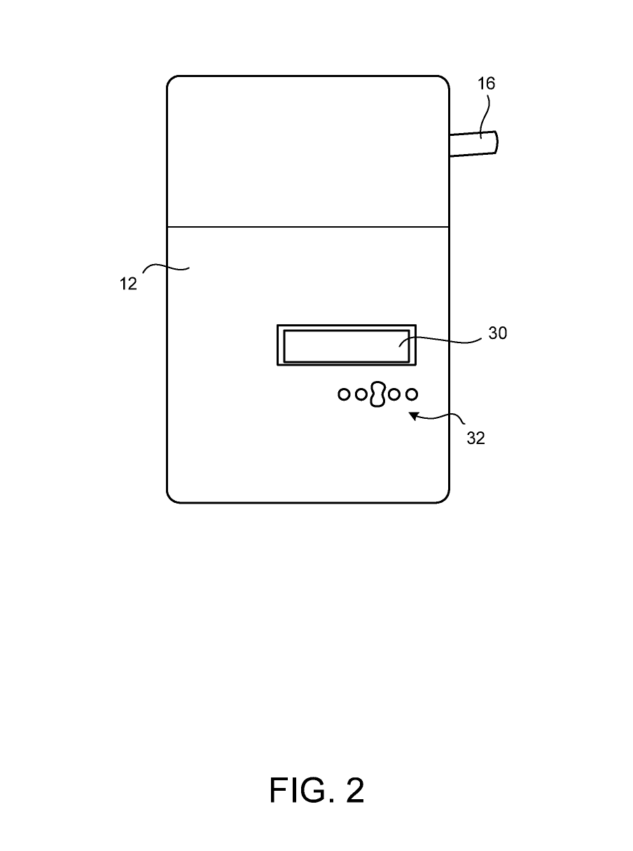

The filter 18, according to one embodiment, is a heat and moisture filter. Accordingly, the filter 18 may be designed and configured to keep the breath chamber 14 at a relatively constant level of temperature and humidity in order to ensure proper sensor output. In one embodiment, the filter 18 is the Thermovent 600 Heat and Moisture Exchanger by Portex. The filter 18 may exhibit low resistance to flow and high moisture filtration in order to minimize the introduction of heat and moisture into the breath chamber 14 through the patient's breath 40. The filter 18 utilizes ISO standard tapered ends in order to ensure a consistent leak-free seal. In one embodiment, the filter 18 is a static, passive element that is replaceable after a certain number of uses. In another embodiment, the filter 18 is an active element that can be controlled by input from the user and/or by input from a predetermined control program. In one embodiment (see FIG. 2), the device 10 includes a control interface (e.g., buttons, switches, touch-screen, etc.) that allows the user to control the operation of the device 10.

In one embodiment, the filter 18 is positioned inside the breath chamber 14 in series with the inlet mouthpiece 16. As the patient breathes into the device 10, the breath 40 travels through the filter 18 and enters the breath chamber 14. The filter 18 is positioned with the patient end proximal to the outside of the shell and the device end facing toward the inside of the breath chamber 14. In one embodiment, the filter 18 meets high standards of quality while remaining price-efficient. In one embodiment, the filter 18 is replaceable after approximately one hundred uses.

The outlet valve 20 can be configured to permit air to exit the breath chamber 14 through the outlet valve 20 and to prevent air from entering the breath chamber 14 through the outlet valve 20. In some embodiments, the outlet valve 20 may be an AlcoMate Breathalyzer Mouthpiece similar to the inlet mouthpiece but positioned differently. In one embodiment, like the inlet mouthpiece 16, the outlet valve 20 is designed to provide one-directionality of flow. According to one embodiment, the valve 20 is positioned inside the breath chamber 14 opposite to the inlet mouthpiece 16 and the filter 18. The patient end of the outlet valve 20 protrudes into the breath chamber 14 while the outlet end is positioned inside a hole leading out of the breath chamber 14. This arrangement allows air to be forced out of the breath chamber 14 during exhalation into the device 10 so that the exhaled breath is able to replace ambient air inside the device 10. Once exhalation stops and the exhaled air has completely replaced any ambient air within the breath chamber 14, the pressure difference across the two ends of the valve 20 will be zero and the check valve mechanism inside the outlet valve 20 will close. This ensures that exhaled air remains in the breath chamber 14 during the entirety of the testing measurement period. Like the inlet mouthpiece 16, the outlet valve 20 may be cost efficient. However, in one embodiment the outlet valve 20 does not need to be replaced as often as the inlet mouthpiece 16 and should instead be replaced any time the filter 18 is exchanged/replaced.

The biometric device 10 may further include a controller 26. In one embodiment, the controller may include an Atmega328PU microcontroller system known as the Arduino Uno. Such a controller is a simple microcontroller system that includes a data output interface (e.g., a USB port), hardware, power management hardware, and clock signal generation hardware on a computer board. In one embodiment, a total of four different peripheral components are connectable to the microprocessor: the power supply, both sensors, and a USB cable used for connecting the device to a personal computer. In one embodiment, the data output interface may be, for example, a USB port that enables the biometric device 10 to be linked with a computer when operating in a computer-linked mode. Via the data output interface, the controller may send data to a linked computer to be recorded, stored, analyzed, displayed, communicated, etc.

The biometric device 10 may further include an integrated power supply 28 (e.g., one or more batteries, capacitors, or the like) or at least a power supply interface that is configured to receive power from an external power supply. In one embodiment, the power supply may be electric and the biometric device may further include electric components that modulate the electricity delivered to the various components of the device 10.

In one embodiment, the display 30 may be an LCD or LED screen that displays testing values and user selectable menus. As described above, the display 30 may be a touchscreen and may function as the user interface through which values and reports are displayed to the user and through which the user actuates or controls the operation of the biometric device. In one embodiment, the LCD or LED screen connects directly to the microprocessor to allow the microprocessor output to be easily displayed for the user to see. In one specific embodiment, the screen includes a 2×16 LCD display and 6 momentary push buttons that are used to navigate through the testing menus. The LCD screen may be powered through its connection to the microprocessor/power source and may operate at 5 VDC. In one embodiment, the display 30 may be black and white or may be multi-colored, thereby providing contrasting backgrounds and easy visibility in a variety of testing situations (outside, inside, day, night, etc).

As described above, the device 10 may be configured to run in a “standard mode” or a “computer-linked mode”. For example, in the standard mode the device 10 may be operated independent of other display elements and/or controllers. In other words, the microprocessor 26 of the device 10 may, in conjunction with the sensors 22, 24, collect the breath data and perform the breath analysis to determine a certain health score (i.e., a certain health condition or a health indicator). The data and/or determined health score may be displayed on the integrated display 30. In the computer-linked mode, according to one embodiment, the biometric device 10 is operated in conjunction with a linked computer. For example, the controller of the device 10 maybe coupled, via a USB port or other similar interface, to a computer or server. In such an embodiment, the breath related data collected via the sensors and/or the health score determined by the device 10 may be displayed, stored, recorded, analyzed, or otherwise manipulated by the linked computer.

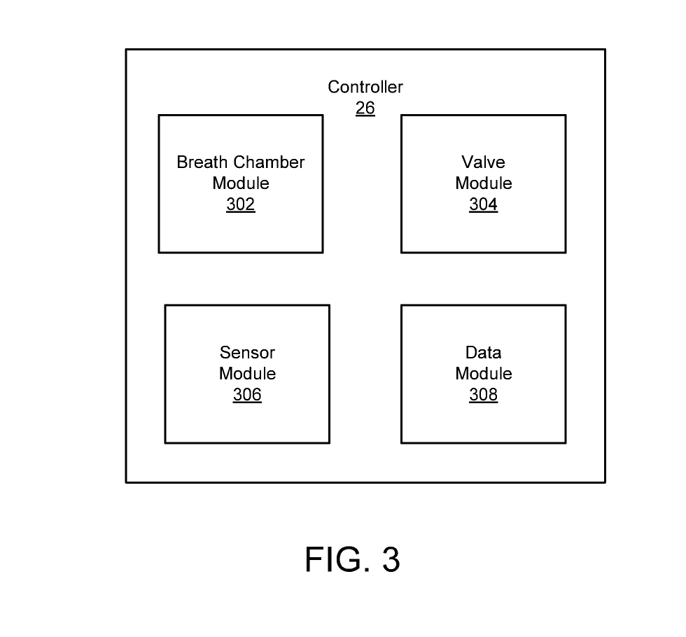

FIG. 3 is a schematic block diagram of one embodiment of the controller 26. The controller 26 includes various modules for determining the heath score of a user that breathes into the device 10. The controller 26, according to one embodiment, includes a breath chamber module 302, valve module 304, a sensor module 306, and a data module 308. The breath chamber module 302, in communication with one or more sensors or dependent on user actuation of a switch/button, indicates the status of the breath chamber 14. In other words, the breath chamber module 302 determines and indicates whether a breath 40 from the user is entering the breath chamber 14, whether the breath 40 from the user is within the breath chamber 14, whether the breath 40 from the user is exiting the breath chamber 14, or whether the breath chamber 14 has no breath 40 contained therein (e.g., standard/baseline atmospheric conditions are present within the breath chamber).

The valve module 304 monitors and optionally controls the one or more valves disposed in the inlet and outlet of the breath chamber. The valve module 304 may simply monitor if the inlet and/or outlet valve is open, thus verifying the reliability of any sensor data collected by the sensors 22, 24. For example, the valve module 304 may indicate a successful sensor data collection if, during the data collection step, the valves sufficiently isolated the breath chamber from the ambient atmosphere external the breath chamber. In other words, the valve module 304 ensures no breath 40 inadvertently or prematurely leaked from the breath chamber 14 and also ensures no external contaminants entered the breath chamber 14 during the sensing step.

The sensor module 306, as described above, receives the sensor data from the sensors 22, 24 and determines the presence and concentration of one or more gases within the breath of the user. The detected concentrations are then communicated to the data module 308 that analyzes the concentrations and determines the health score or the health indicator of the user. The controller 26 may also include additional modules, such as a tracking module that tracks the user's health scores over time, thus enabling the user to determine how certain activities or treatments are affecting the user's health score. Such feedback is very beneficial in maximizing the user's ability to improve his/her health.

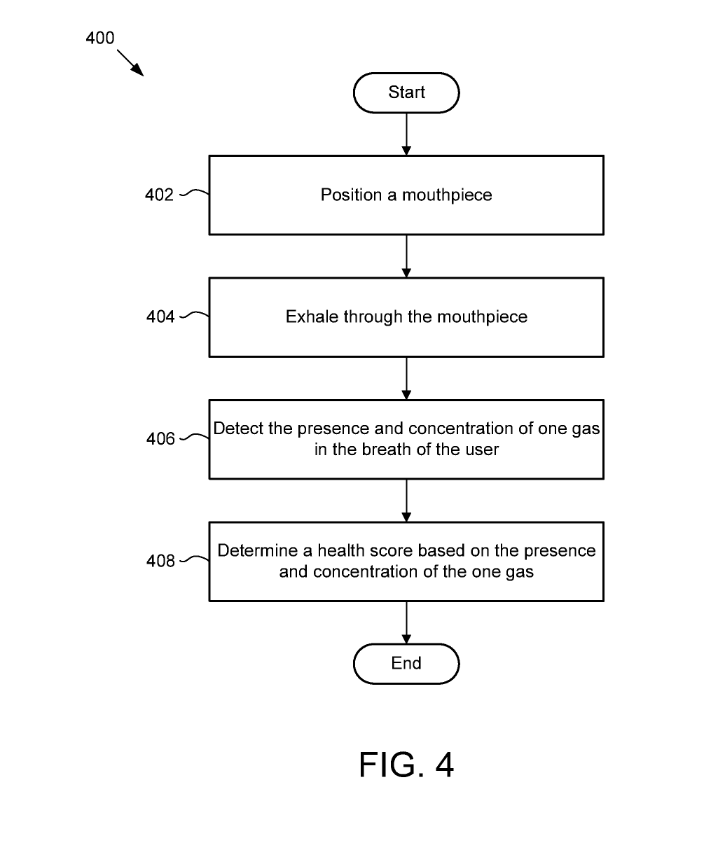

FIG. 4 is a schematic flow chart diagram of one embodiment of a method 400 for determining the health score of a user. The method 400 includes positioning 402 a mouthpiece of a biometric device into the mouth of the user and exhaling 404 a breath through the mouthpiece an into a breath chamber of the biometric device. The method 400 also includes detecting 406 the presence and concentration of at least one gas in the breath of the user and determining 408 a health score of the user based on the presence and concentration of the at least one gas in the breath of the user. In one implementation, the method includes holding the breath within the breath chamber for a predetermined time before allowing the breath to exit the breath chamber.

The device, system, and method may be used for other purposes (other than health tracking) such as biometric verification and authentication.

As described above, the biometric device 10 may include a single sensor or a plurality of sensors. For example, in one embodiment, the biometric device 10 includes a carbon monoxide sensor 22 and a carbon dioxide sensor 24. These sensors, according to one embodiment, may be sensors manufactured by Alphasense. The carbon monoxide sensor 22 may be positioned within the device housing 12 and adjacent to the breath chamber 14. The carbon monoxide sensor may include a sensing element or a sensing portion that is at least partially disposed within and is in fluid communication with the breath chamber 14. For example, a small borehole or aperture in the wall of the breath chamber may allow a sensing element of the carbon monoxide sensor 22 to take readings relating to the exhaled air that is within the breath chamber. The sensor may, for example, detect the presence and/or concentration of carbon monoxide within the breath chamber 14. In one embodiment, the sensor may operate using chemical reactions to test for and detect the presence of the carbon monoxide. The detected presence/concentration of carbon monoxide may be reported to the controller for further analysis/display.

In one embodiment, the carbon monoxide sensor 22 has a good resolution of less than about 0.5 ppm, thereby allowing the sensor to detect small changes in carbon monoxide in the breath 40 of a user. This sensor may be calibrated to allow for a fairly large dynamic range of 0-100 ppm, which covers the entire range of carbon monoxide for physiological conditions as well as for most diseased states. The carbon monoxide sensor 22 also features a relatively fast response time and is capable of reaching 90 percent of its stable value from zero to 400 ppm carbon monoxide in less than 25 seconds. This allows for multiple tests to be completed in a relatively short amount of time. The carbon monoxide sensor 22 also has a very low hydrogen cross sensitivity. Since hydrogen is relatively abundant in exhaled breath 40, this is an extremely important feature.

Similar to the carbon monoxide sensor 22, the carbon dioxide sensor 24 may be at the top of the main chamber 13 and may include a sensing element or sensing portion that is exposed to the breath chamber 14 to allow readings to be obtained from this chamber during testing. In one embodiment, the sensor operates using optical testing, leading to extremely high signal to noise ratio.

In order to obtain useful and accurate wellness information from the device, the sensors 22 and 24 may first be calibrated using known concentrations of test gases. Once calibrated, the biometric device 10 can be used in one or more of the two modes described above. Though the carbon monoxide and carbon dioxide readings obtained are useful biological health indicators, they are prone to variability due to incomplete filling of the breath chamber during testing or metabolic effects. Therefore, the health score which is given as the ratio of the carbon monoxide and carbon dioxide readings, should instead be used as the final indicator of health. The health score corrects for the variability due to incomplete filling and metabolic effects to produce a more reliable result.

Though the health score determined by the device represents an important indicator of overall health in an absolute sense, the device, according to one embodiment, is more appropriately used to view changes in health over time. In other words, held scores determined by the device over a period of time will indicate a health trend of the user. Therefore, the device of the present disclosure may be used, for example, for daily testing to gauge how certain health activities (ingestion of supplements, exercise, sleep pattern, etc.) impact overall health.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer readable medium may be a tangible computer readable storage medium storing the program code. The computer readable storage medium may be, for example, but not limited to, an electronic, magnetic, optical, electromagnetic, infrared, holographic, micromechanical, or semiconductor system, apparatus, or device, or any suitable combination of the foregoing.

More specific examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store program code for use by and/or in connection with an instruction execution system, apparatus, or device.

The computer readable medium may also be a computer readable signal medium. A computer readable signal medium may include a propagated data signal with program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport program code for use by or in connection with an instruction execution system, apparatus, or device. Program code embodied on a computer readable signal medium may be transmitted using any appropriate medium, including but not limited to wire-line, optical fiber, Radio Frequency (RF), or the like, or any suitable combination of the foregoing

In one embodiment, the computer readable medium may comprise a combination of one or more computer readable storage mediums and one or more computer readable signal mediums. For example, program code may be both propagated as an electro-magnetic signal through a fiber optic cable for execution by a processor and stored on RAM storage device for execution by the processor.

Program code for carrying out operations for aspects of the present invention may be written in any combination of one or more programming languages, including an object oriented programming language such as Java, Smalltalk, C++, PHP or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The computer program product may be shared, simultaneously serving multiple customers in a flexible, automated fashion. The computer program product may be standardized, requiring little customization and scalable, providing capacity on demand in a pay-as-you-go model. The computer program product may be stored on a shared file system accessible from one or more servers.

The computer program product may be integrated into a client, server and network environment by providing for the computer program product to coexist with applications, operating systems and network operating systems software and then installing the computer program product on the clients and servers in the environment where the computer program product will function.

In one embodiment software is identified on the clients and servers including the network operating system where the computer program product will be deployed that are required by the computer program product or that work in conjunction with the computer program product. This includes the network operating system that is software that enhances a basic operating system by adding networking features.

Furthermore, the described features, structures, or characteristics of the embodiments may be combined in any suitable manner. In the following description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described below with reference to schematic flowchart diagrams and/or schematic block diagrams of methods, apparatuses, systems, and computer program products according to embodiments of the invention. It will be understood that each block of the schematic flowchart diagrams and/or schematic block diagrams, and combinations of blocks in the schematic flowchart diagrams and/or schematic block diagrams, can be implemented by program code. The program code may be provided to a processor of a general purpose computer, special purpose computer, sequencer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

The program code may also be loaded onto a computer, other programmable data processing apparatus, or other devices to cause a series of operational steps to be performed on the computer, other programmable apparatus or other devices to produce a computer implemented process such that the program code which executed on the computer or other programmable apparatus provide processes for implementing the functions/acts specified in the flowchart and/or block diagram block or blocks.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A biometric device comprising:
a housing comprising a main chamber and a breath chamber, wherein the breath chamber comprises an inlet and an outlet;
a sensor coupled to the main chamber and at least partially in fluid contact with the breath chamber, the sensor being configured to detect the concentration of at least one gas of a user's breath in the breath chamber; and
a controller coupled to the housing and disposed within the main chamber, the controller configured to receive the concentration of the at least one gas in the user's breath in the breath chamber and determine a health score.

2. The device of claim 1, wherein the at least one gas includes carbon monoxide.

3. The device of claim 2, wherein the at least one gas further includes carbon dioxide.

4. The device of claim 1, further comprising an inlet valve and a moisture filter coupled to the inlet of the breath chamber of the housing, wherein both the inlet valve and the moisture filter are configured to be in fluid communication with the breath of the user.

5. The device of claim 4, wherein the inlet valve is a one way valve that prevents the breath from exiting the breath chamber through the inlet.

6. The device of claim 1, wherein the outlet comprises an outlet valve, wherein the outlet valve is a one way valve that prevents atmosphere from entering the breath chamber through the outlet.

7. The device of claim 1, wherein the main chamber and the breath chamber of the housing are detachable from each other.

8. The device of claim 1, wherein the controller comprises a breath chamber module configured to indicate a status of the breath chamber.

9. The device of claim 1, wherein the controller comprises a valve module configured to indicate a status of valves disposed in the inlet and outlet of the breath chamber.

10. The device of claim 1, wherein the controller comprises a sensor module configured to determine the concentration of the at least one gas of the breath of the user in the breath chamber.

11. The device of claim 10, wherein the controller comprises a data module configured to determine the health score based on the concentration of the at least one gas of the breath of the user.

12. The device of claim 1, wherein the controller comprises:
a breath chamber module configured to indicate a status of the breath chamber;
a valve module configured to indicate a status of valves disposed in the inlet and outlet of the breath chamber;
a sensor module configured to determine the concentration of the at least one gas of the breath of the user in the breath chamber; and
a data module configured to determine the health score based on the concentration of the at least one gas of the breath of the user.

13. A biometric system comprising:
a housing comprising a main chamber and a breath chamber, wherein the breath chamber comprises an inlet and an outlet;
a sensor coupled to the main chamber and at least partially in fluid contact with the breath chamber, the sensor being configured to detect the concentration of at least one gas of a user's breath in the breath chamber; and
a controller comprising:
a breath chamber module configured to indicate a status of the breath chamber;
a valve module configured to indicate a status of valves disposed in the inlet and outlet of the breath chamber;
a sensor module configured to determine the concentration of the at least one gas of the breath of the user in the breath chamber; and
a data module configured to determine the health score based on the concentration of the at least one gas of the breath of the user.

14. The system of claim 13, wherein the controller is integrated on a computer removed from the housing.

15. The system of claim 13, further comprising an inlet valve and a moisture filter coupled to the inlet of the breath chamber of the housing, wherein both the inlet valve and the moisture filter are configured to be in fluid communication with the breath of the user.

16. The system of claim 15, wherein the inlet valve is a one way valve that prevents the breath from exiting the breath chamber through the inlet.

17. The system of claim 13, wherein the outlet comprises an outlet valve, wherein the outlet valve is a one way valve that prevents atmosphere from entering the breath chamber through the outlet.

18. The system of claim 13, wherein the main chamber and the breath chamber of the housing are detachable from each other.

19. A method for determining a health score of a user, the method comprising:
positioning a mouthpiece of a biometric device into the mouth of the user;
exhaling a breath through the mouthpiece an into a breath chamber of the biometric device;
detecting the presence and concentration of at least one gas in the breath of the user; and
determining a health score of the user based on the presence and concentration of the at least one gas in the breath of the user.

20. The method of claim 19, further comprising holding the breath within the breath chamber for a predetermined time before allowing the breath to exit the breath chamber.
